(19) United States
(12) Patent Application Publication
(10) Pub. No.: US 2021/0290956 A1
(43) Pub. Date: Sep. 23, 2021

(54) ON-CHIP PACEMAKER CELLS FOR ESTABLISHING AN AUTONOMOUSLY CONTROLLABLE ELECTRICAL PACEMAKER

(71) Applicant: Universität Rostock, Rostock (DE)

(72) Inventors: Robert David, Wittenbeck (DE); Christian Rimmbach, Lichtenhagen (DE); Julia Jung, Lichtenhagen (DE)

(21) Appl. No.: 17/260,309

(22) PCT Filed: Jul. 17, 2019
(86) PCT No.: PCT/EP2019/069196

§ 371 Date: Jan. 14, 2021

(30) Foreign Application Priority Data
Jul. 18, 2018 (DE) 10 2018 212 005.6

(51) Int. Cl. A61N 1/365; C12N 5/077; A61N 1/372; A61N 1/375

(52) U.S. Cl. CPC A61N 1/365; A61N 1/37512; A61N 1/37217; C12N 5/0657

(57) ABSTRACT

The present invention relates to a bioelectronic system comprising at least i) in vitro generated cardiac pacemaker tissue comprising cardiac pacemaker cells; ii) an electronic circuit applied to a semiconductor carrier and comprising at least one field-effect transistor; wherein the in vitro generated cardiac pacemaker tissue comprising cardiac pacemaker cells according to i) is immobilized on the electronic circuit according to ii) applied to the semiconductor carrier and is arranged in conductive communication with the at least one field-effect transistor. The invention also relates to a rate-adaptive cardiac pacemaker system comprising at least one rate-adaptive cardiac pacemaker, comprising at least one sensor unit, wherein the sensor unit is set up to detect at least one electrical cardiac signal; at least one pulse generator, wherein the pulse generator is set up to generate at least one electrical pacemaker pulse and to deliver it to the heart of a patient; at least one control unit, wherein the control unit is electrically connected to the sensor unit and the pulse generator; a bioelectronic system, wherein the bioelectronic system is conductively connected or connectable to the rate-adaptive cardiac pacemaker, in particular to the control unit.

The present invention relates to a bioelectronic system comprising at least i) in vitro generated cardiac pacemaker tissue comprising cardiac pacemaker cells; ii) an electronic circuit applied to a semiconductor carrier and comprising at least one field-effect transistor; wherein the in vitro generated cardiac pacemaker tissue comprising cardiac pacemaker cells according to i) is immobilized on the electronic circuit according to ii) applied to the semiconductor carrier and is arranged in conductive communication with the at least one field-effect transistor. The invention also relates to a rate-adaptive cardiac pacemaker system comprising at least one rate-adaptive cardiac pacemaker, comprising at least one sensor unit, wherein the sensor unit is set up to detect at least one electrical cardiac signal; at least one pulse generator, wherein the pulse generator is set up to generate at least one electrical pacemaker pulse and to deliver it to the heart of a patient; at least one control unit, wherein the control unit is electrically connected to the sensor unit and the pulse generator; a bioelectronic system, wherein the bioelectronic system is conductively connected or connectable to the rate-adaptive cardiac pacemaker, in particular to the control unit.

A cardiac pacemaker is an electronic pulse generator that is used to electrically stimulate the heart muscle to contract in order to compensate for cardiac arrhythmias. Natural cardiac pacemakers are the sinus node and sometimes also the atrioventricular node. Artificial cardiac pacemakers are electronic devices which are usually implanted and which also function as electronic pulse generators. By contrast with the natural pacemaker sinus node, electronic cardiac pacemakers currently do not respond to autonomous stimulation such as for example physical stress or psychological excitement, so the heart rate is not adjusted by electronic cardiac pacemakers to these physiological states of the body.

The functional coupling of nerve cells to semiconductor chips is known in principle and is described for example by Fromherz in the 2009 research report of the Max Planck Institute for Biochemistry. Various approaches for the generation of cardiac pacemaker cells are also known, for example via forward programming from multipotent or pluripotent stem cells (see WO 2015/091157 A1, WO 2017/108895 A1), by means of direct programming from embryonic stem cells, or by means of reprogramming from for example cardiomyocytes (WO 2013/070952 A1). US 2003/0036773 A1 describes a bioelectronic cardiac pacemaker system with a field-effect transistor.

An object on which the present invention is based was therefore that of providing a cardiac pacemaker or components suitable for this purpose capable of overcoming the essential disadvantages of conventional electronic pacemakers.

It has surprisingly been found that a bioelectronic system can be used to provide such a cardiac pacemaker or its components in which in vitro generated cardiac pacemaker tissue comprising cardiac pacemaker cells is immobilized on an electronic circuit applied to a semiconductor carrier and is arranged in conductive communication with at least one field-effect transistor.

The invention therefore relates to a bioelectronic system, comprising at least
i) in vitro generated cardiac pacemaker tissue comprising cardiac pacemaker cells;
ii) an electronic circuit applied to a semiconductor carrier and comprising at least one field-effect transistor;
   wherein the in vitro generated cardiac pacemaker tissue comprising cardiac pacemaker cells according to i) is immobilized on the electronic circuit according to ii) applied to the semiconductor carrier and is arranged in conductive communication with the at least one field-effect transistor.

In the context of the present invention, a field-effect transistor (FET) is basically understood as meaning a functional unit which comprises at least one source electrode, at least one drain electrode and at least one gate electrode. Furthermore, the field-effect transistor comprises at least one source-drain channel, wherein a current can flow between the source electrode and the drain electrode through the source-drain channel, at least under specific external conditions. These conditions may comprise in particular a voltage applied to the source-drain channel or an electrical potential applied to the source-drain channel. In this case, the electrical potential may be applied to the source-drain channel in particular by means of the gate electrode described in more detail below or by means of an external electrode. The source-drain channel may comprise at least one semiconductor material, in particular a doped semiconductor material. The gate electrode may comprise at least one liquid solution, wherein the solution may comprise at least one electrolyte. An FET comprising such a gate electrode may also be referred to as a “liquid-gated FET”. Furthermore, the source-drain channel may have an insulating layer, in particular comprising at least one oxide layer, wherein the insulating layer may at least partially electrically isolate the source-drain channel from the gate electrode.

According to a preferred embodiment of the bioelectronic system, the in vitro generated cardiac pacemaker tissue comprising cardiac pacemaker cells is generated from myocardial cells, preferably by means of reprogramming. The direct reprogramming may take place, for example, from ventricular cardiomyocytes, possibly using TBX factors such as for example TBX18. A corresponding reprogramming is disclosed by way of example in example 1 of WO 2013/070952 A1, the disclosure of which is incorporated here by reference.

In one embodiment of the bioelectronic system, the in vitro generated cardiac pacemaker tissue comprising cardiac pacemaker cells is generated from embryonic stem cells, preferably by means of direct programming, more preferably by means of direct programming using Shox2. Corresponding direct programming is disclosed for example in example 4 of WO 2013/070952 A1, which is incorporated here by reference.

In one embodiment of the bioelectronic system, the in vitro generated cardiac pacemaker tissue comprising cardiac pacemaker cells is generated from multipotent or pluripotent stem cells, preferably from pluripotent stem cells, preferably by means of forward programming (induced sinoatrial cell bodies (iSABs) comprising cardiac pacemaker cells). Here, sinus node cells (cardiac pacemaker cells) are preferably generated from stem cells, in which a nucleic acid is introduced into stem cells, whereby they express a TBX transcription factor, or a TBX protein is introduced into the stem cells, wherein a construct for expressing an antibiotic resistance gene, which is controlled by an alpha-MHC (MYH6) promoter, is additionally introduced and the resulting stem cells are differentiated in the presence of the antibiotic. This generation of sinus node cells is described in WO 2015/091157 A1, the disclosure of which is incorporated herein by reference. In particular, in this embodiment the in vitro generated cardiac pacemaker tissue comprising cardiac pacemaker cells is generated from multipotent or pluripotent stem cells, preferably from pluripotent stem cells, wherein at least one TBX transcription factor, in particular TBX3, is used in combination with an antibiotic selection on the basis of the Myh6 promoter.

If a nucleic acid for the expression of a TBX transcription factor is introduced into the stem cells, it is preferably selected from TBX DNA, in particular TBX cDNA; or TBX-RNA, in particular TBX-mRNA. Within the framework of the RNA, TBX-mRNA may be transfected into the stem cells, but this does not result in a stable gene modification. Alternatively, microRNAs that bring about expression of endogenous TBX may be introduced. In a preferred embodiment of the nucleic acid introduction, TBX-DNA, in particular TBX-cDNA, is introduced by means of a vector, in particular by means of an (over)expression vector. The TBX is preferably selected from TBX3 or TBX-18, with TBX3 being particularly preferred and TBX3 cDNA being highly preferred. In other words, a highly preferred variant is the introduction of TBX3-cDNA with an overexpression vector. With regard to the TBX protein, which likewise does not cause a (stable) gene modification, TBX3 is likewise preferred. Human or non-human nucleic acids or proteins are used, with those of human origin being preferred.

With regard to the stem cells used, multipotent or pluripotent, preferably pluripotent, stem cells are used. The stem cells may be selected from human or non-human embryonic stem cells or human or non-human induced stem cells or human induced stem cells or parthenogenetic stem cells or spermatogonial stem cells. The cardiac pacemaker tissue comprising cardiac pacemaker cells is preferably generated from non-human embryonic stem cells or non-human induced pluripotent stem cells or human induced pluripotent stem cells or parthenogenetic stem cells or spermatogonial stem cells, preferably by means of forward programming, particularly preferably from non-human embryonic stem cells or non-human induced pluripotent stem cells or human induced pluripotent stem cells. In this preferred and particularly preferred variant, human embryonic stem cells are explicitly excluded.

The antibiotic selection on the basis of the Myh6 promoter preferably uses an antibiotic resistance gene selected from the aminoglycoside antibiotic resistance gene, more preferably from the neomycin and puromycin resistance gene, highly preferably from the neomycin resistance gene. The antibiotic used for the selection is appropriately selected from an aminoglycoside antibiotic, in particular from neomycin and puromycin. “Appropriately selected” means that the antibiotic that matches the resistance gene is always used; for example, with the neomycin resistance gene, neomycin is then used for selection.

Cardiac pacemaker cells (human or non-human) are generated and appropriately used in each case, with human cardiac pacemaker cells being preferred. For this purpose, human stem cells are combined with preferably human protein or human nucleic acid. Cross combinations, such as for example the introduction of human proteins or human nucleic acids into non-human, for example murine, stem cells is likewise possible, as is the pure combination of the non-human representatives for generating non-human cardiac pacemaker cells.

The in vitro generated cardiac pacemaker tissue comprising cardiac pacemaker cells is preferably at least partially in electrical contact with a gate electrode of the field-effect transistor.

The gate electrode preferably comprises at least one electrolyte, in particular the gate electrode consists of a solution comprising the at least one electrolyte.

In a preferred embodiment of the bioelectronic system, an electrical potential applied to the gate electrode can be influenced directly or indirectly by an electrical primary signal generated by the in vitro generated cardiac pacemaker tissue comprising cardiac pacemaker cells. In the context of the present invention, an “electrical primary signal” is basically understood as meaning any electrical signal that is generated on the basis of a previous physiological event and/or on the basis of at least one incoming physiological signal, in particular a molecule, for example a hormone. The electrical primary signal generated by the in vitro generated cardiac pacemaker tissue comprising cardiac pacemaker cells comprises at least one ion current, in particular an ion current including potassium ions and/or calcium ions. The ion current generated by the in vitro generated cardiac pacemaker tissue comprising cardiac pacemaker cells can thus flow into the solution of the gate electrode and the charge ratios of the solution of the gate electrode, and consequently the electrical potential applied to the source-drain channel or the voltage applied to the source-drain channel, can change. In particular, the ion current can change the electrical potential applied to the source-drain channel or the voltage applied to the source-drain channel in such a way that the source-drain channel becomes electrically conductive for a current between the source electrode and the drain electrode, as described in more detail below.

In a preferred embodiment of the bioelectronic system, the electrical primary signal generated by the in vitro generated cardiac pacemaker tissue comprising cardiac pacemaker cells can be influenced by a physiological factor of a vicinity of the in vitro generated cardiac pacemaker tissue comprising cardiac pacemaker cells. In particular, it may be possible that a physiological factor can cause or suppress generation of the electrical primary signal or influence, in particular determine, a property of the electrical primary signal, such as for example a signal strength or a signal frequency of a sequence of electrical primary signals. A positive chronotropic effect (increase in frequency) can be achieved by stimulating the sympathetic system through the transmitters adrenaline and noradrenaline, which reach the target cells via the bloodstream. A negative chronotropic effect (decrease in frequency) results from parasympathetic stimulation by means of acetylcholine via the vagus nerve.

In one embodiment of the bioelectronic system, the gate electrode is in direct or indirect physical contact with at least one source-drain channel of the field-effect transistor. The gate electrode may thus be separated from the source-drain channel in particular by the insulating layer, in particular the oxide layer. The gate electrode may however also be in direct physical contact with the source-drain channel.

An electrical response signal can preferably be generated in the source-drain channel by the electrical primary signal by means of influencing the electrical potential applied to the gate electrode. In the context of the present invention, an “electrical response signal” is basically understood as meaning any electrical signal that arises or is generated as a reaction to an electrical signal that is present. In particular, the electrical response signal may be a current signal, for example a current flowing from the source electrode to the drain electrode through the source-drain channel. As already described, the electrical primary signal, for example in the form of the ion current, can change the electrical potential applied to the source-drain channel or the voltage applied to the source-drain channel in such a way that the source-drain channel becomes electrically conductive for the current signal between the source electrode and the drain electrode. With a suitable voltage applied between the source electrode and the drain electrode, the electrical primary signal of the in vitro generated cardiac pacemaker tissue comprising cardiac pacemaker cells can in this way generate an electrical response signal in the source-drain channel. The source-drain channel preferably comprises at least one semiconducting layer. Further preferably, the semiconducting layer of the source-drain channel comprises at least one material selected from the group consisting of: an element semiconductor, in particular silicon; a compound semiconductor; an organic semiconductor.

The invention also relates to a rate-adaptive cardiac pacemaker system comprising at least
a rate-adaptive cardiac pacemaker, comprising
at least one sensor unit, wherein the sensor unit is set up to detect at least one electrical cardiac signal;
at least one pulse generator, wherein the pulse generator is set up to generate at least one electrical pacemaker pulse and to deliver it to the heart of a patient;
at least one control unit, wherein the control unit is electrically connected to the sensor unit and the pulse generator;
a bioelectronic system as described above, wherein the bioelectronic system is conductively connected or connectable to the rate-adaptive cardiac pacemaker, in particular to the control unit.

In the context of the present invention, a sensor unit is basically understood as meaning any device which is set up to quantitatively or qualitatively detect, register, measure, receive, record or pass on an electrical signal, in particular a voltage signal or a current signal.

In the context of the present invention, a pulse generator is basically understood as meaning any device which is set up to generate at least one electrical signal, in particular a voltage signal and/or a current signal. In particular, the pulse generator for generating the electrical pulse, for example the electrical pacemaker pulse, may comprise a pair of electrodes.

In the context of the present invention, a control unit is basically understood as meaning any electronic device which is set up to execute, activate or evaluate at least one function of the rate-adaptive cardiac pacemaker system or the rate-adaptive cardiac pacemaker. The control unit may have in particular at least one measuring device and/or at least one electrical energy source. Furthermore, the control unit may have at least one processor or circuit which can perform an evaluation function of the electrical cardiac signal detected by the sensor unit and/or the electrical response signal generated by the bioelectronic system. Furthermore, the processor or the circuit may be set up to perform a control function of the pulse generator.

In particular, the pulse generator may be set up to generate a sequence of electrical pacemaker pulses and to deliver them to the heart of a patient;

In one embodiment of the rate-adaptive cardiac pacemaker system, the electronic circuit of the bioelectronic system is set up to pass on to the control unit at least one response signal generated by an electrical primary signal of the in vitro generated cardiac pacemaker tissue comprising cardiac pacemaker cells in a source-drain channel of a field-effect transistor of the bioelectronic system.

In a preferred embodiment, the control unit is set up to measure a time interval between at least two successive response signals. Furthermore, the control unit may be set up to measure a time interval between at least two successive electrical cardiac signals. Furthermore, the control unit may be set up to compare the time interval between the at least two successive response signals with the time interval between the at least two successive electrical cardiac signals. Furthermore, the control unit may be set up to generate at least one electrical pacemaker pulse by means of the pulse generator and to deliver it to the heart of a patient if the time interval between the at least two successive electrical cardiac signals exceeds the time interval between the at least two successive response signals.

In particular, the time interval between the at least two successive electrical cardiac signals may exceed, in particular significantly exceed, the time interval between the at least two successive response signals. The control unit may therefore be set up to replace the time interval between the at least two successive electrical cardiac signals with a substitution value if a measurement duration of the time interval between the at least two successive electrical cardiac signals exceeds a specified threshold value, wherein the substitution value exceeds the time interval between at least two successive response signals. In particular, the specified threshold value may be the time interval between the at least two successive response signals. Furthermore, the control unit may be set up to generate at least one electrical pacemaker pulse by means of the pulse generator and to deliver it to the heart of a patient if the substitution value exceeds the time interval between the at least two successive response signals.

In one embodiment of the rate-adaptive cardiac pacemaker system, the control unit is set up to specify for the pulse generator the at least one time interval between the at least two successive electrical pacemaker pulses, taking into account the at least one electrical cardiac signal and the at least one response signal.

Furthermore, the rate-adaptive cardiac pacemaker system may comprise an energy source, a storage medium and an external component, wherein the external component may be set up to receive data from the rate-adaptive cardiac pacemaker, in particular from the control unit, by means of wireless transmission. Furthermore, the rate-adaptive cardiac pacemaker system may also comprise further elements not mentioned here.

The present invention is illustrated in more detail by the following embodiments and combinations of embodiments that arise from the corresponding dependency references and other references. In particular, it should be noted here that in every case in which a range of embodiments is named, for example in the context of an expression such as “bioelectronic system according to one of the embodiments 1 to 4”, every embodiment in this range is meant to be explicitly disclosed to the person skilled in the art, i.e. for the person skilled in the art, the formulation of this expression is to be understood as synonymous with “bioelectronic system according to one of the embodiments 1, 2, 3 and 4”.

### EMBODIMENTS

1. A bioelectronic system, comprising at least
i) in vitro generated cardiac pacemaker tissue comprising cardiac pacemaker cells;
ii) an electronic circuit applied to a semiconductor carrier and comprising at least one field-effect transistor;
   wherein the in vitro generated cardiac pacemaker tissue comprising cardiac pacemaker cells according to i) is immobilized on the electronic circuit according to ii) applied to the semiconductor carrier and is arranged in conductive communication with the at least one field-effect transistor.

2. The bioelectronic system according to embodiment 1, wherein the in vitro generated cardiac pacemaker tissue comprising cardiac pacemaker cells is generated from myocardial cells, preferably by means of reprogramming.

3. The bioelectronic system according to embodiment 1, wherein the in vitro generated cardiac pacemaker tissue comprising cardiac pacemaker cells is generated from embryonic stem cells, preferably by means of direct programming.

4. The bioelectronic system according to embodiment 1, wherein the in vitro generated cardiac pacemaker tissue comprising cardiac pacemaker cells is generated from multipotent or pluripotent stem cells, preferably from pluripotent stem cells, preferably by means of forward programming (induced sinoatrial cell bodies (iSABs) comprising cardiac pacemaker cells).

5. The bioelectronic system according to embodiment 4, wherein the induced sinoatrial cell bodies (iSABs) comprising cardiac pacemaker cells are generated from non-human embryonic stem cells or non-human induced pluripotent stem cells or human induced pluripotent stem cells or parthenogenetic stem cells or spermatogonial stem cells, preferably by means of forward programming, preferably from non-human embryonic stem cells or non-human induced pluripotent stem cells or human induced pluripotent stem cells.

6. The bioelectronic system according to one of the embodiments 1 to 5, wherein the in vitro generated cardiac pacemaker tissue comprising cardiac pacemaker cells is at least partially in electrical contact with a gate electrode of the field-effect transistor.

7. The bioelectronic system according to embodiment 6, wherein the gate electrode comprises at least one electrolyte, in particular consists of a solution comprising the at least one electrolyte.

8. The bioelectronic system according to one of the embodiments 6 or 7, wherein an electrical potential applied to the gate electrode can be influenced directly or indirectly by an electrical primary signal generated by the in vitro generated cardiac pacemaker tissue comprising cardiac pacemaker cells.

9. The bioelectronic system according to embodiment 8, wherein the electrical primary signal generated by the in vitro generated cardiac pacemaker tissue comprising cardiac pacemaker cells comprises at least one ion current, in particular an ion current comprising potassium ions and/or calcium ions.

10. The bioelectronic system according to one of the embodiments 8 or 9, wherein the electrical primary signal generated by the in vitro generated cardiac pacemaker tissue comprising cardiac pacemaker cells can be influenced by a physiological factor of a vicinity of the in vitro generated cardiac pacemaker tissue comprising cardiac pacemaker cells.

11. The bioelectronic system according to one of the embodiments 8 to 10, wherein the gate electrode is in direct or indirect physical contact with at least one source-drain channel of the field-effect transistor.

12. The bioelectronic system according to embodiment 11, wherein an electrical response signal can be generated in the source-drain channel by the electrical primary signal by means of influencing the electrical potential applied to the gate electrode.

13. The bioelectronic system according to one of the embodiments 11 or 12, wherein the source-drain channel comprises at least one semiconducting layer.

14. The bioelectronic system according to embodiment 13, wherein the semiconducting layer of the source-drain channel comprises at least one material selected from the group consisting of: an element semiconductor, in particular silicon; a compound semiconductor; an organic semiconductor.

15. A rate-adaptive cardiac pacemaker system comprising at least
a rate-adaptive cardiac pacemaker, comprising
   at least one sensor unit, wherein the sensor unit is set up to detect at least one electrical cardiac signal;
   at least one pulse generator, wherein the pulse generator is set up to generate at least one electrical pacemaker pulse and to deliver it to the heart of a patient;
   at least one control unit, wherein the control unit is electrically connected to the sensor unit and the pulse generator;
a bioelectronic system according to one of the embodiments 1 to 14, wherein the bioelectronic system is conductively connected or connectable to the rate-adaptive cardiac pacemaker, in particular to the control unit.

16. The rate-adaptive cardiac pacemaker system according to embodiment 15, wherein the electronic circuit of the bioelectronic system is set up to pass on to the control unit at least one response signal generated by an electrical primary signal of the in vitro generated cardiac pacemaker tissue comprising cardiac pacemaker cells in a source-drain channel of a field-effect transistor of the bioelectronic system.

17. The rate-adaptive cardiac pacemaker system according to embodiment 16, wherein the control unit is set up to specify for the pulse generator the at least one time interval between the at least two successive electrical pacemaker pulses, taking into account the at least one electrical cardiac signal and the at least one response signal.

### CITED LITERATURE

Peter Fromherz, Research Report 2009 of the Max Planck Institute for Biochemistry

WO 2015/091157 A1

WO 2017/108895 A1

WO 2013/070952 A1

US 2003/0036773 A1

1. A bioelectronic system, comprising at least
i) in vitro generated cardiac pacemaker tissue comprising cardiac pacemaker cells;
ii) an electronic circuit applied to a semiconductor carrier and comprising at least one field-effect transistor;
wherein the in vitro generated cardiac pacemaker tissue comprising cardiac pacemaker cells according to i) is immobilized on the electronic circuit according to ii) applied to the semiconductor carrier and is arranged in conductive communication with the at least one field-effect transistor.

2. The bioelectronic system as claimed in claim 1, wherein the in vitro generated cardiac pacemaker tissue comprising cardiac pacemaker cells is produced from myocardial cells, preferably by means of reprogramming.

3. The bioelectronic system as claimed in claim 1, wherein the in vitro generated cardiac pacemaker tissue comprising cardiac pacemaker cells is generated from multipotent or pluripotent stem cells (induced sinoatrial cell bodies (iSABs) comprising cardiac pacemaker cells).

4. The bioelectronic system as claimed in claim 1, wherein the in vitro generated cardiac pacemaker tissue comprising cardiac pacemaker cells is at least partially in electrical contact with a gate electrode of the field-effect transistor.

5. The bioelectronic system as claimed in claim 4, wherein an electrical potential applied to the gate electrode can be influenced directly or indirectly by an electrical primary signal generated by the in vitro generated cardiac pacemaker tissue comprising cardiac pacemaker cells.

6. The bioelectronic system as claimed in claim 5, wherein the electrical primary signal generated by the in vitro generated cardiac pacemaker tissue comprising cardiac pacemaker cells can be influenced by a physiological factor of a vicinity of the in vitro generated cardiac pacemaker tissue comprising cardiac pacemaker cells.

7. The bioelectronic system as claimed in claim 5, wherein the gate electrode is in direct or indirect physical contact with at least one source-drain channel of the field-effect transistor, wherein an electrical response signal can be generated in the source-drain channel by the electrical primary signal by means of influencing the electrical potential applied to the gate electrode.

8. The bioelectronic system as claimed in claim 7, wherein the source-drain channel comprises at least one semiconducting layer, wherein the semiconducting layer of the source-drain channel comprises at least one material selected from the group consisting of: an element semiconductor; a compound semiconductor; and an organic semiconductor.

9. A rate-adaptive cardiac pacemaker system comprising at least
a rate-adaptive cardiac pacemaker, comprising
at least one sensor unit, wherein the sensor unit is set up to detect at least one electrical cardiac signal;
at least one pulse generator, wherein the pulse generator is set up to generate at least one electrical pacemaker pulse and to deliver it to the heart of a patient;
at least one control unit, wherein the control unit is electrically connected to the sensor unit and the pulse generator;
a bioelectronic system as claimed in claim 1, wherein the bioelectronic system is conductively connected or connectable to the rate-adaptive cardiac pacemaker.

10. The rate-adaptive cardiac pacemaker system as claimed in claim 9, wherein the electronic circuit of the bioelectronic system is set up to pass on to the control unit at least one response signal generated by an electrical primary signal of the in vitro generated cardiac pacemaker tissue comprising cardiac pacemaker cells in a source-drain channel of a field-effect transistor of the bioelectronic system.
